(19) United States
(12) Patent Application Publication
(10) Pub. No.: US 2021/0188051 A1
(43) Pub. Date: Jun. 24, 2021

(54) IDENTIFYING AND MITIGATING VEHICLE ODORS

(71) Applicant: Ford Global Technologies, LLC, Dearborn, MI (US)

(72) Inventors: Perry Robinson MACNEILLE, Dearborn, MI (US); Victoria Leigh SCHEIN, Dearborn, MI (US); Jeffrey Brian YEUNG, Dearborn, MI (US); Clay Wesley MARANVILLE, Dearborn, MI (US); Kerrie Kathleen GATH, Dearborn, MI (US); Daniel BOCCUCCIA, Dearborn, MI (US)

(21) Appl. No.: 16/754,202

(22) PCT Filed: Oct. 13, 2017
(86) PCT No.: PCT/US2017/056660

§ 371 Date: Apr. 7, 2020

(51) Int. Cl. B60H 3/00; G06F 16/906; G06F 16/9035

(52) U.S. Cl. CPC B60H 3/0085; G06F 16/9035; G06F 16/906

(57) ABSTRACT

A method for mitigating odor includes detecting a known smell using on one or more odor sensors in a vehicle. The method includes determining whether the known smell is agreeable to one or more passengers of the vehicle. The method includes mitigating the known smell using one or more odor control devices if the known smell is not agreeable to the one or more passengers of the vehicle.

### TECHNICAL FIELD

The disclosure relates generally to identifying and mitigating odors in vehicles.

### BACKGROUND

Odors and fragrances within a vehicle can significantly contribute to or detract from a passenger's comfort and enjoyment during travel. As automated vehicles and vehicle sharing becomes more common, identification and mitigation of odors may become more challenging and more difficult.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1 is a schematic diagram illustrating an operating environment for an odor mitigation system, according to one implementation;

FIG. 2 is a schematic block diagram illustrating interconnections between an odor mitigation system and other sensors or data sources, according to one implementation;

FIG. 3 is a schematic block diagram illustrating components and interconnections of a mobile communication device, according to one implementation;

FIG. 4 is a schematic block diagram illustrating components of an odor mitigation system, according to one implementation;

FIG. 5 a schematic flow chart diagram illustrating a method for mitigating an odor or smell, according to one implementation;

FIG. 6 a schematic flow chart diagram illustrating a method for processing sensor data and mitigating odors based on passenger preferences, according to one implementation;

FIG. 7 is a schematic flow chart diagram illustrating a method for learning a mitigation strategy during attempting mitigation of an odor or smell, according to one implementation;

FIG. 8 is a schematic flow chart diagram illustrating a method for determining a user's preferences with regard to a smell, according to one implementation;

FIG. 9 is a schematic flow chart diagram illustrating a method for learning a new mitigation strategy while eliminating an odor and retaining the fragrances, according to one implementation; and

FIG. 10 is a schematic flow chart diagram illustrating a method for interaction between an odor mitigation system and a mobile communication device of an occupant, according to one implementation; and

FIG. 11 is a schematic block diagram illustrating a computing system, according to one implementation.

### DETAILED DESCRIPTION

Odor control is an important future differentiator between vehicles in all markets. In “mobility as a service” activities where many people are sharing vehicles or using vehicle owned by someone else, odor control becomes even more important and more complex solutions are needed. Strategies for odor control or mitigation may include controlling odor precursors, removing the source of the odor, dilution of the odor, emission of fragrances, biological or chemical transformation to destroy or change odor causing particles or chemicals, chemical binding to remove odor causing particles or chemicals, masking of odors, and/or olfactory desensitization. Vehicles may have passive and active controls for cabin odors such as windows which may be opened or closed, HVAC (heating, ventilation, and air condition) recirculation control, HVAC dehumidification, sources for air fragrances, systems for adding water ions to the air, systems for air filtration and particle/chemical absorbance using antimicrobial materials, absorbents, ultraviolet light, or the like.

While some existing odor control systems may be quite complex, Applicants have recognized the need to automate odor control systems and techniques using artificial intelligence and other technologies to make them more effective, easier and less distracting to use. Odor mitigation can be quite complex because there is a lack of models that comprehensively relate psychological responses to odor with the chemistry that produce them. The problem is likely to become even more complex as new sensing systems evolve and more specific mitigation approaches evolve. Furthermore, response to odors can be very specific to individuals and may vary based on cultural, geographic, demographic, or other backgrounds. The response to odors is frequently time dependent. That is odor introduced gradually may cause no response to humans. Furthermore, a cascade or series of odors may be indicative of a particular source or event.

At least one embodiment disclosed herein may provide for automatic means of odor control. Other embodiments may provide advice or notify a user of steps that can be taken to perform odor control or mitigation. For example, a driver of a vehicle who is planning to pick up a passenger may be reminded to remove a source of an odor, apply a fragrance, or ventilate the vehicle before picking up the passenger.

One of the best odor sensors in a vehicle are the occupants or passengers. Even if cabin sensors do not detect an odor, the occupants may. The complexity of describing an odor may require a spoken dialog system to communicate between an occupant and an odor mitigation system. However, occupants may provide individualized descriptions of odors if no common descriptive language exists. Thus, embodiments may utilize phrases or terms provided by occupants, even unique to a specific occupant, based on occupant responses.

In some embodiments, a system may encounter a new odor which does not have a known mitigation strategy, at least for a specific vehicle. If the sensor fingerprint (e.g., odor characteristics as detected by odor sensors) and an occupant confirms the existence of an odor, the system may need to explore mitigation strategies to find one that is effective. For example, if skunk smell is detected it may be best to close the windows as quickly as possible to prevent the odor from entering the vehicle. If it is cigarette smoke, the opposite may be the case and vehicle systems bring fresh air into the cabin. By learning what steps have contributed to odor mitigation previously or in different vehicles, systems may improve and share odor mitigation strategies.

Embodiments disclosed herein may include systems, methods, and devices for identifying odors in a vehicle, learning or storing an odor's chemistry, learning or storing a human language label for the odor, and/or identifying or sharing odor mitigation strategies. In at least one embodiment, a method for odor mitigation may include detecting a known smell using on one or more odor sensors in a vehicle. The method may include determining whether the known smell is agreeable to one or more passengers of the vehicle. The method may include mitigating the known smell using one or more odor control devices if the known smell is not agreeable to the one or more passengers of the vehicle. Embodiments may allow for an odor mitigation system to ensure that a vehicle always smells good. “Good” in this case is a qualitative term that can mean different things to different occupants. For example, at least one embodiment learns an occupants' preferences and removes smells or adds fragrance discriminately based on a history of dialog with the occupants. In some cases, a vehicle or even a mobile device of an occupant may log preferences specific to the vehicle or occupant.

At least some embodiments provide for learning capabilities. For example, an odor mitigation system may learn various aspects of odor management. Example aspects of odor management may include how to use odor management devices on a vehicle to control a specific odor, how to interpret sensor data to determine identify odors in the air, and a descriptive language for odors used by occupants in general or for a specific occupant. Training for these aspects may be both collaborative and occur during real world usage. Occupants may have a personal preference database stored on a mobile device that they carry into each vehicle. The vehicle may have a vehicle database that contains rules and adjustments for peculiarities of the individual vehicle because different vehicles may have different geometries, HVAC systems, sensors, mitigation devices, controllers, or the like.

An odor mitigation system may implement a spoken dialog system to allow it to interact with the occupants of a vehicle and process input data from vehicle occupants. The odor mitigation system monitors the changes in sensor data or measurements and may look for patterns that would indicate a known smell. If the recommender detects a known smell, it determines whether the individuals in the vehicle consider the smell to be disagreeable (an odor) or agreeable (a fragrance). The odor mitigation system may poll the individual preference databases in each mobile device in the vehicle and determine whether mitigation of the smell is necessary based on a “vote”.

If there is a smell in the vehicle that the odor mitigation system is unfamiliar with, a vehicle occupant may report it to the odor mitigation system. The odor mitigation system will try to identify the pattern of sensor measurements that are consistent with the odor. The odor mitigation system may connect to other odor mitigation systems, and if the smell is known, information about it is downloaded from the other odor mitigation system and mitigation begins if necessary. If the smell is unknown even to other odor mitigation system the odor mitigation system may use its resources to learn occupant's responses to the odor in the vehicle, how it can be identified, and how it can be mitigated. Information about smells can be exchanged using a vehicle-to-vehicle (V2V) or a mobile communication network.

The vehicle sensors used to detect smells and provide input to the odor mitigation system may be specific gas sensors, arrays of specific gas sensors, sensors that measure physical properties of the air in the vehicle (e.g., air temperature, pressure, humidity, particulate size or count, etc.). Odor mitigation devices or odor control devices may include the vehicle's windows, blower, HVAC doors, devices that inject chemicals into the air, generate ions, dehumidify the air, remove chemicals from the air, use static electric charge, emit light (ultraviolet light), move air, and/or devices that modify the source of smells such as anti-microbial filtering devices.

For the purposes of this disclosure, smells result from a combination of micro-components of the air that produce an olfactory or other physiological stimulus and a psychological response to occupants. Odors are smells that produce a disagreeable response while fragrances are smells that produce an agreeable response to occupants.

Further embodiments and examples will be discussed in relation to the figures below.

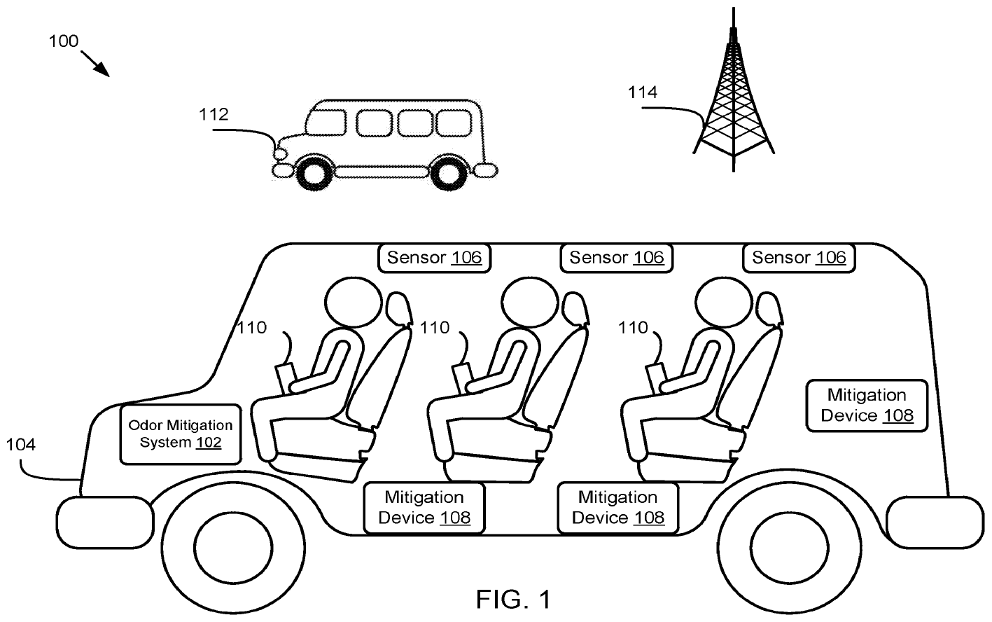

FIG. 1 is a schematic diagram illustrating an operating environment 100 for an odor mitigation system 102, according to one embodiment. The operating environment illustrates a vehicle 104 carrying a plurality of occupants. The vehicle 104 includes the odor mitigation system 102 (such as within an in-dash computing system), a plurality of smell sensors 106, a plurality of odor mitigation devices 108, and mobile communication devices 110 of the occupants. The odor mitigation system 102 may be in wired or wireless communication with the sensors 106, mitigation devices 108, and/or mobile communication devices 110. The odor mitigation system 102 may also communicate with another vehicle 112 (or vehicles) or any other device directly or via a mobile network tower 114 (e.g., over the Internet)).

The sensors 106 may include an electrochemical nose (e-nose) with an array of sensors. An electrochemical nose may include sensors and implement algorithms to identify the presence or combination of chemicals or other attributes of air within the vehicle. The sensors 106 or the electrochemical nose may include one or more of a molecular sensor, chemosensor, gas chromatography sensors or other sensor for smell classification. Examples of specific sensor technologies which may be used include conductive-polymer odor sensors, tin-oxide gas sensors, quartz-crystal micro-balance sensors, capacitive micromachined ultrasonic transducers (CMUT), or the like.

The mitigation devices 108 may include any type of device for adding, moving, or removing air from a vehicle interior. Other mitigation devices 108 may include sources for fragrances, chemicals for reacting to chemical components of odors, air filters or purifiers, or the like. For example, window controllers, HVAC systems, filtration systems, air freshener systems, or the like may be examples of mitigation devices 108. In one embodiment, a mitigation device 108 may include a plurality chemical sources that can be used to selectively emit chemicals or compounds into the air that will react with different odor components or chemicals. For example, one source may emit a first chemical that reacts with a first type of odor causing chemical while a second source may emit a second chemical that reacts with a different type of odor causing chemical.

The odor mitigation system 102 may be implemented as a computing system having one or more processors and storage readable media in communication with the processor(s). The odor mitigation system 102 may identify and mitigate odors in the vehicle 104 based on data from the sensors 106, mobile communication devices 110, other vehicles 112, or any other source. The odor mitigation system 102 may maintain a database containing user preferences regarding odors and odor mitigation. In some situations, the odor mitigation system 102 may remove odors or add fragrances based on known user preferences (e.g., preferences stored by the odor mitigation system 102 or provided by the mobile communication devices 110). Additionally, the odor mitigation system 102 may communicate with other vehicles or systems to resolve odor problems within the vehicle 104.

In one embodiment, the odor mitigation system 102 is capable of learning how to mitigate odors over time by using odor mitigation devices 108 to control a specific odor, how to interpret sensor inputs to identify odors, and to develop a descriptive language (spoken by human users) for odors. In some situations, a particular odor is identified by a user, but the odor mitigation system 102 is not familiar with mitigating the odor. To mitigate the odor, the odor mitigation system 102 in the specific vehicle communicates with other odor mitigation systems in other vehicles (e.g., vehicle 112) to receive odor mitigation information related to the identified odor. The received information is then used to mitigate the odor in the vehicle 104 and the information is stored by the odor mitigation system 102 for future reference.

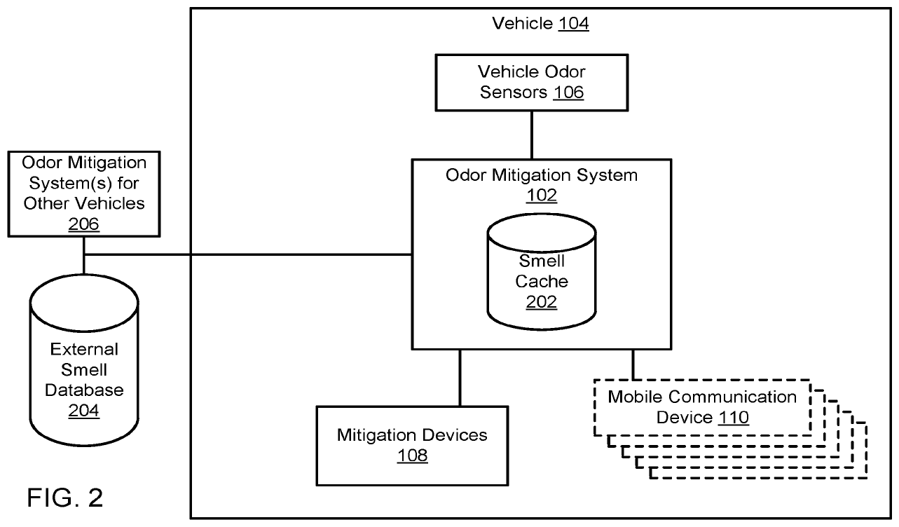

FIG. 2 is a schematic block diagram illustrating interconnections between an odor mitigation system and other sensors or data sources. The odor mitigation system 102 may include a smell cache 202 that stores information about a smell such as the sensor attributes corresponding to the smell, the preferences of one or more users with regard to the smell, human language or terms associated with the smell, and/or mitigation procedures for mitigating or removing the smell (odor). The odor mitigation system 102 may communicate with mobile communication devices 110 of occupants to receive their preferences with regard to a smell. The odor mitigation system 102 may also access an external smell database 204 that is located remotely from the vehicle 104, such on a server accessible via the Internet. The external smell database may include information specific to the vehicle 104, occupants of the vehicle, the type of vehicle corresponding to the vehicle 104, and/or smell data (e.g., preferences) for the general population. The odor mitigation system 102 may also communicate with odor mitigation systems 206 for other vehicles to obtain smell preferences, mitigation procedures, or the like.

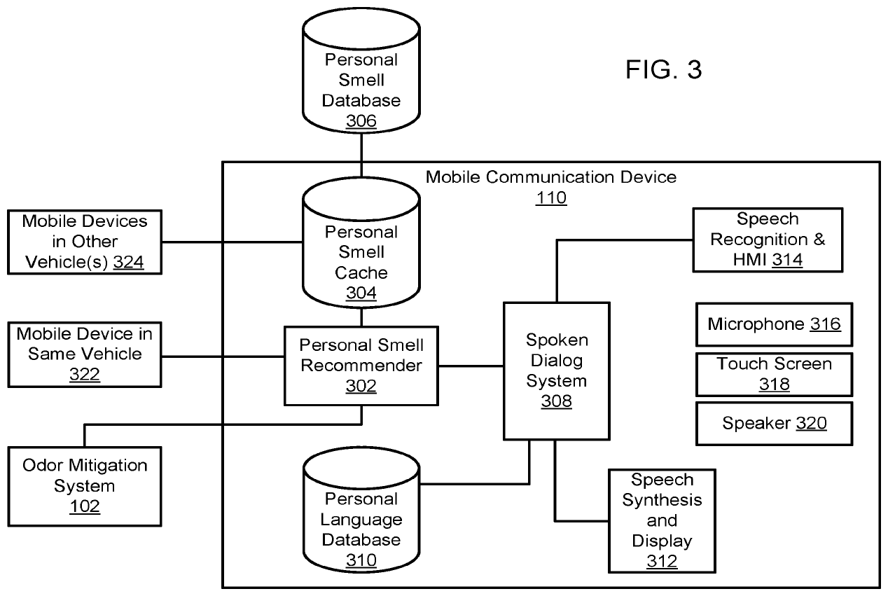

FIG. 3 is a schematic block diagram illustrating components and interconnections of a mobile communication device 110, according to one embodiment. For example, the mobile communication device 110 may include a smartphone, tablet, or other computing device with storage, hardware, and/or installed programs or applications (e.g., computer code or instructions) that make up various components of the mobile communication device 110. A personal smell recommender 302 may provide recommendations as to which smells qualify as odors or fragrances for an owner of the mobile communication device 110. The personal smell recommender 302 may access data in a personal smell cache 304 local to the mobile communication device 110 and/or a personal smell database 306 local or remote from the mobile communication device 110. The personal smell cache 304 and/or the personal smell database 306 may store smells encountered by the user of the mobile communication device 110 as well as the user's preferences with regard to those smells. In one embodiment, a user may only require his or her preferences for a smell once and that preference is then stored in the personal smell cache 304 and/or the personal smell database 306.

A spoken dialog system 308 allows a user to speak to and receive verbal instructions or queries from the mobile communication device 110. The spoken dialog system 308 may access a personal language database 310 that is specific to a user so that smells can be described in terms that the user understands. For example, the spoken dialog system 308, upon the mobile communication device 110 detecting a new smell (e.g., based on information provided by an odor mitigation system 102 of a vehicle), may ask the user to describe the smell and to indicate whether they like or dislike the smell, as well as how severe or strong the smell is. The spoken dialog system 308 may then update the personal language database 310 to include terms that correspond to the new smell as well as log the user's preferences in the personal smell cache 304 and the personal smell database 306. If the user doesn't have any specific preferred terms for a smell, the spoken dialog system 308 may use default terms or terms obtained from other users' devices to get the conversation started and update the terms as the user provides a description. In one embodiment, the personal language database 310 may be included within a shared database that includes the information for personal language database 310, personal smell cache 304, and personal smell cache 306. The spoken dialog system 308 and a speech and syntheses and display system 312 may interact with the user using a speech recognition and a human machine interface (HMI) 314. For example, the speech recognition and HMI 314 may use a microphone 316, touch screen 318, speaker 320, or any other input or output devices to interact with or receive input from a user. In one embodiment, the speech and syntheses and display system 312, HMI 314, microphone 316, screen 318, and speaker 320 may be part build into a vehicle, such as part of a vehicle infotainment system or in-dash computing system which can be accessed using a user's mobile device. For example, a user may link or communicate with the in-dash computing system or vehicle infotainment system using Ford® SmartDeviceLink™ or other system or software.

The mobile communication device 110 may provide personal smell preferences to the odor mitigation system 102 of a vehicle in which a user is currently an occupant. The mobile communication device 110 may receive descriptions or indications of a current smell (e.g., in human language or in chemical or physical property descriptions of the air) in the vehicle from the odor mitigation system 102 and respond with the user's preferences or response to the smell. In one embodiment, if a new smell is encountered the mobile communication device 110 may prompt the user for their input or response, while if it is a known smell (e.g., has an entry in the personal smell cache or personal smell database) the mobile communication device 110 may simply provide the user's preferences without bothering or querying the occupant.

The mobile communication device 110 may communicate with mobile communication devices 322, 324 in the same or different vehicles to obtain information about encountered smells, human descriptions of smells, or the like. For example, as a user travels between vehicles, the mobile communication device 110 may obtain information about smells that others have encountered so that the mobile communication device 110 may provide a guess as to the user's preferences or language which the user will understand. Having this information may speed up the process for the user in understanding a query about a smell and/or providing the user's own specific preferences with respect to that smell.

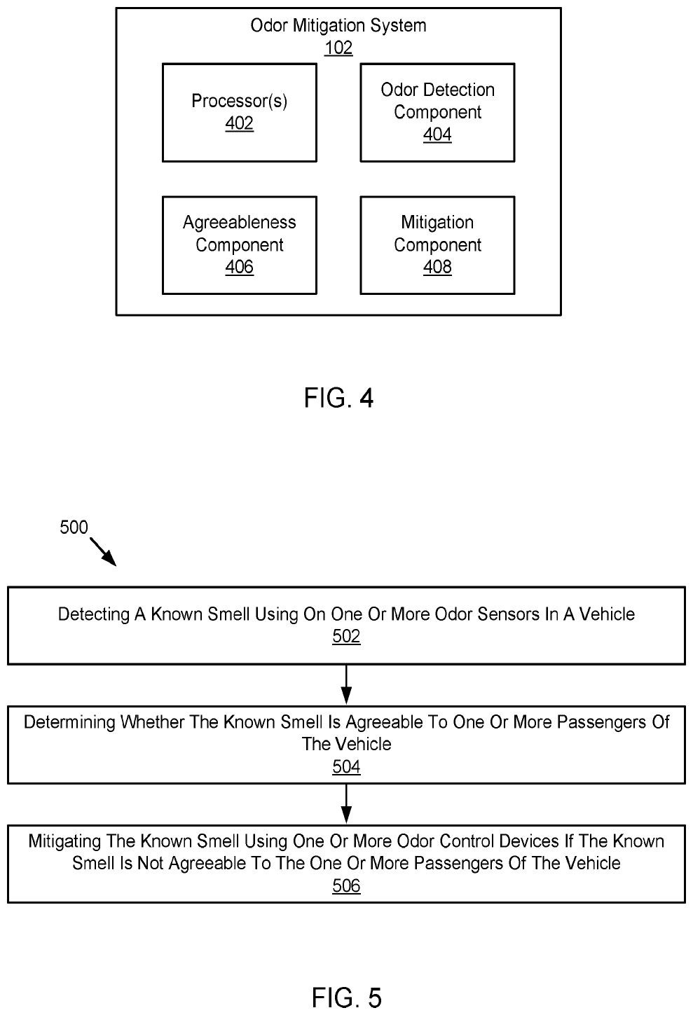

FIG. 4 is a schematic block diagram illustrating some components of an odor mitigation system 102, according to one embodiment. The odor mitigation system 102 includes one or more processors 402, an odor detection component 404, an agreeableness component 406, and a mitigation component 408. The components 402-408 are given by way of illustration only and may not all be included in all embodiments. In fact, some embodiments may include only one or any combination of two or more of the components 402-408. For example, some of the components 402-408 may be located outside or separate from the odor mitigation system 102. Furthermore, the components 402-408 may include hardware, processors, computer readable instructions, or a combination of both to perform the functionality and provide the structures discussed herein.

The processors 402 may include any type of processors or processing circuits. In one embodiment, the processors 402 may include a conventional desktop, laptop, or server central processing unit (CPU). In one embodiment, the processors 402 may include multiple parallel processors such as those found in a graphics processing unit (GPU), accelerated processing unit (APU), or neural processing unit (NPU). Parallel processing may be helpful for performing the computations required by a neural network.

The odor detection component 404 is configured to detect a known smell based on the one or more odor sensors. For example, the odor detection component 404 may receive sensor outputs or sensor signatures provided by odor sensors. The odor detection component 404 may detect the known smell by matching signals or parameters from the one or more odor sensors with attributes of a smell logged in a smell database or smell cache. The odor detection component 404 may also match information from the one or more odor sensors with a description provided by a human. The odor detection component 404 may match sensor data to a known odor based on chemical signatures, physical air properties, or any other sensor data.

The agreeableness component 406 is configured to determine whether the known smell is agreeable to one or more passengers of the vehicle. For example, the agreeableness component 406 communicates a chemical, human language, or other description to the one or more passengers or their personal devices. The personal devices or passengers may then respond to indicate how the passengers perceive the smell. The agreeableness component 406 may determine whether the known smell is agreeable to the one or more passengers based on an indication received from a mobile device for each of the one or more passengers. For example, each passenger or device may provide a “vote” for whether they find the smell agreeable (fragrance) or disagreeable (odor) as well as how severe the odor is. An indication may include a preference of a specific passenger stored on a mobile communication device or a response by the passenger to a query about the known smell.

The mitigation component 408 is configured to control one or more odor control devices to mitigate the known smell if the known smell is not agreeable to the one or more passengers of the vehicle. For example, the mitigation component 408 may send signals to any mitigation device such as an HVAC system, window, chemical source, fragrance source, or the like, to mask, remove, or otherwise reduce an odor. The mitigation component 408 may identify a mitigation procedure for mitigating a known or unknown smell or odor. For example, the mitigation component 408 may identify the mitigation procedure by identifying a known smell in a mitigation database, the mitigation database indicating a mitigation procedure for the known smell. As another example, if it is an unknown smell or there is no known mitigation procedure, the mitigation component 408 may query a remote database or other vehicles for mitigation procedures. In one embodiment, a default mitigation procedure may be used in cases where no specific mitigation procedure for the smell can be obtained from local or remote sources. One of a plurality of default mitigation procedures may be selected and tried. By tracking how the smell reacts, based on sensor data and user perceptions, the mitigation component 408 may learn how best to handle the new smell and share that with others. In one embodiment, the mitigation component 408 uses a mitigation database (which may be part of a smell cache or smell database) that includes a database specific to the vehicle, wherein the database specific to the vehicle indicates adjustments to mitigation procedures based on specific attributes of the vehicle. The mitigation database may include a shared database shared by a plurality of vehicles, wherein the shared database matches one or more mitigation procedures from another vehicle with the known smell.

FIG. 5 a schematic flow chart diagram illustrating a method 500 for mitigating an odor or smell, according to one embodiment. For example, the method 500 may be performed by an odor mitigation system 102, such as the odor mitigation systems of FIG. 1, 2, or 4. The method begins and an odor detection component 404 detects 502 a known smell using on one or more odor sensors in a vehicle. An agreeableness component 406 determines 504 whether the known smell is agreeable to one or more passengers of the vehicle. For example, each device or passenger may provide a “vote” indicating whether the odor is agreeable or disagreeable as well as how severe or strong the odor is. The agreeableness component 406 may determine 504 whether to treat the smell as an odor or fragrance based on these responses. A mitigation component 408 mitigates 506 the known smell using one or more odor control devices if the known smell is not agreeable to the one or more passengers of the vehicle.

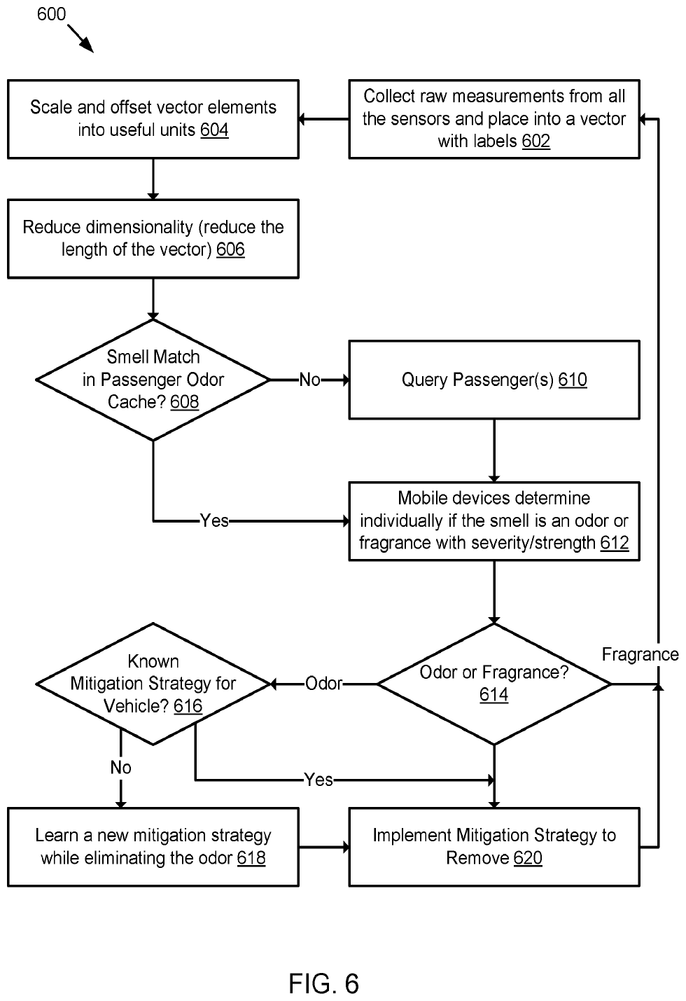

FIG. 6 a schematic flow chart diagram illustrating a method 600 for processing sensor data and mitigating odors based on passenger preferences, according to one embodiment. For example, the method 600 may be performed by an odor mitigation system 102 and/or a mobile communication device 110. The odor mitigation system 102 collects 602 or samples raw measurements from all the sensors in the vehicle and place the measurements (e.g., sample distributions) into a vector with labels. The measurements may indicate a value for a measured parameter such as temperature, air pressure, particle count, humidity, the presence and amount of a chemical or compound, or any other measurement values. The odor mitigation system 102 scales and offsets 604 vector elements into useful units. For example, the measurements may be converted to a desired format or type (such as a random variable) or may be scaled by a multiplier. For example, the measurements may be offset by an augend and then marshalled into a vector of chemical activity values. The odor mitigation system 102 reduces 606 the dimensionality of the vector by removing or combining duplicative measurements (e.g., distributions using feature extraction) into a single measurement within the vector. The odor mitigation system 102 may forward the resulting vector to any occupant mobile communication device 110 to get their preferences.

Each mobile communication device 110 may check 608 to see if the resulting vector has a matching smell or odor in a personal cache or database. If there is no matching odor or smell (No at 608), the mobile communication device 110 may query 610 the passenger or user (e.g., occupant) of the device for their preference on the smell. Based on the response, the mobile communication device 110 determines 612 if the smell is an odor or fragrance as well as the severity or strength of the smell. If there is a matching odor or smell (Yes at 608), the mobile communication device 110 may determine 612 if the smell is an odor or fragrance as well as the severity or strength of the smell based on data stored in a personal smell database or personal smell cache.

Each mobile communication device 110 may provide a corresponding occupants' preferences and the odor mitigation system 102 determines 614 whether the smell represented by the vector is an odor or fragrance. If the smell is a fragrance (Fragrance at 614), the odor mitigation system 102 ignores it and returns to collecting 602 sensor measurements. If the smell is an odor (Odor at 614), the odor mitigation system 102 determines 616 whether there is a known mitigation strategy for the odor in the current vehicle. If there is a known mitigation strategy (Yes at 616), the odor mitigation system 102 implements 620 the mitigation strategy to remove the odor. If there is not a known mitigation strategy (No at 616), the odor mitigation system 102 learns 618 a new mitigation strategy specific to the odor while eliminating the odor. For example, the odor mitigation system 102 may implement a default mitigation strategy or obtain a strategy from another vehicle or online database. The odor mitigation system 102 may learn 618 by implementing 620 the strategy and tracking how well the odor is removed based on sensor measurements and/or occupant's perception of a reduction, increase, or no change in the smell. Upon mitigation of the smell (e.g., after implementing 620 the mitigation strategy) the odor mitigation system 102 may return to collecting 602 sensor measurements.

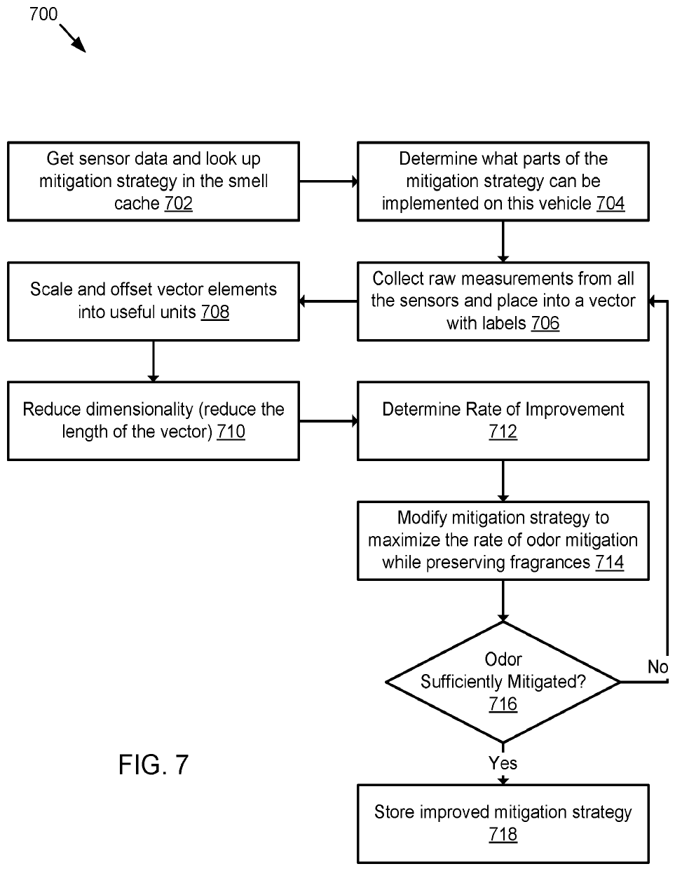

FIG. 7 is a schematic flow chart diagram illustrating a method 700 for learning a mitigation strategy during attempting mitigation of an odor or smell, according to one embodiment. The method 700 may be performed, for example, by an odor mitigation system 102.

The odor mitigation system 102 may get 702 sensor data and look up a mitigation strategy, if any, in the smell cache (e.g., a personal, vehicle specific, or remote cache or database). The odor mitigation system 102 determines 704 what parts of the mitigation strategy can be implemented on a current vehicle. The odor mitigation system 102 collects 706 raw measurements from all the sensors and places them into a vector with labels, scales and offsets 708 the vector elements into useful units, and reduces 710 dimensionality of the vector. The odor mitigation system 102 determines 712 a rate of improvement of the smell. The rate of improvement may be determined 712 by additional sensor measurements, querying the user or user device after mitigation has begun, or the like. The odor mitigation system 102 modifies 714 the mitigation strategy to maximize the rate of odor mitigation while preserving fragrances. For example, the odor mitigation system 102 may identify mitigation steps or portions of a mitigation procedure that led to the fastest odor mitigation or may periodically introduce a random mitigation procedure and measure how the rate of improvement changes. The odor mitigation system 102 determines 716 whether the odor is sufficiently mitigated 716, such as by taking additional sensor measurements or querying an occupant or device. If the odor is sufficiently mitigated (Yes at 716), the odor mitigation system 102 stores 718 the (improved) mitigation strategy for later recall when this odor is encountered. If the odor is not sufficiently mitigated (No at 716), the odor mitigation system 102 may begin again to collect 706 sensor data and further modify or track the mitigation.

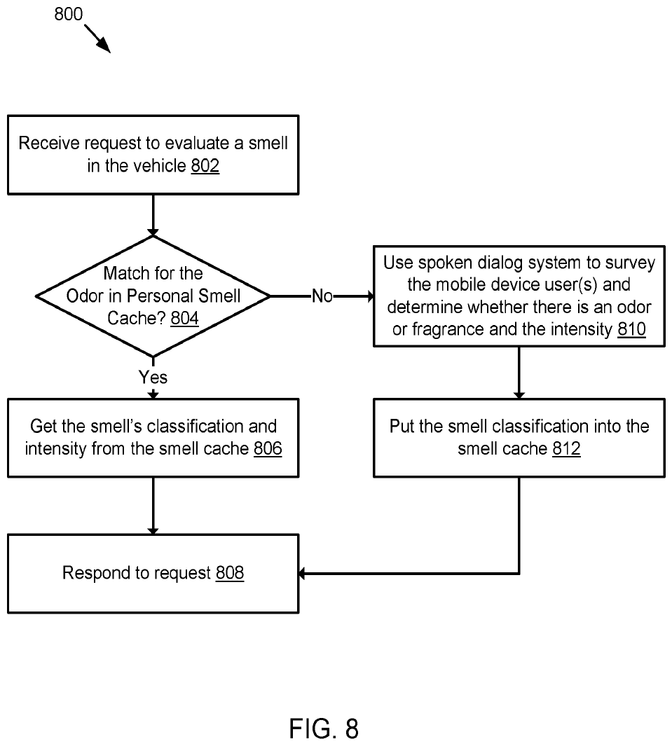

FIG. 8 is a schematic flow chart diagram illustrating a method 800 for determining a user's preferences with regard to a smell, according to one embodiment. The method 800 may be performed, for example, by a mobile communication device 110 of an occupant of a vehicle.

The mobile communication device 110 receives 802 a request to evaluate a smell in the vehicle. For example, the mobile communication device 110 may receive 802 the request from an odor mitigation system 102 of a vehicle. The request may include a description of the smell of interest, such as a vector including sensor measurements and/or human language labels. The mobile communication device 110 determines 804 whether there is a match for the smell in a personal smell cache corresponding to the mobile communication device 110 or user of the mobile communication device 110. For example, the mobile communication device 110 may search a database for an entry that corresponds to a description received in the request. If there is a match (Yes at 804), the mobile communication device 110 gets 806 the smell's classification and intensity from a smell cache for the user (e.g., stored locally or remotely from the mobile communication device 110) and then responds 808 to the request with the classification (e.g., odor or fragrance) and intensity (e.g., on a scale from 1-10). If there is not a match (No at 804), the mobile communication device 110 uses a spoken dialog system to survey 810 an occupant using the mobile communication device 110 to determine whether a smell is detected by the occupant, whether the smell is an odor or fragrance for that occupant, and/or how the occupant would rate the intensity of the smell. The user's responses, including any terms the user used during the dialog, may be put 812 in a smell cache for later retrieval and responds 808 to the request with the classification and intensity.

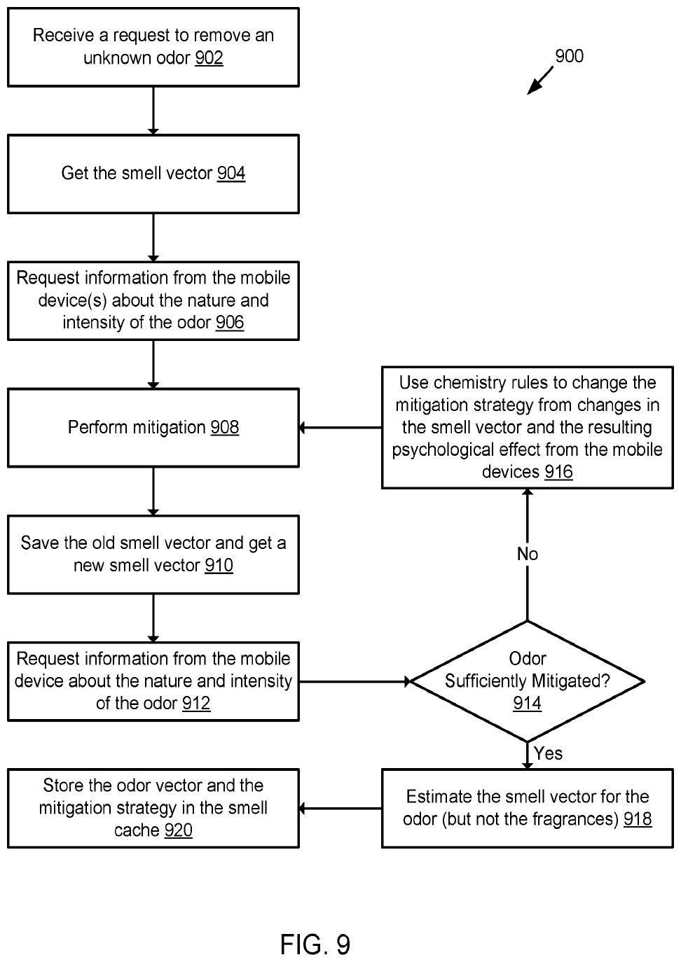

FIG. 9 is a schematic flow chart diagram illustrating a method 900 for learning a new mitigation strategy while eliminating an odor and retaining the fragrances, according to one embodiment. The method 900 may be performed by an odor mitigation system 102, for example.

The odor mitigation system 102 receives 902 a request to remove an unknown odor. For example, an occupant's device may send a request to remove an odor with a description of the odor. The odor may include a human language description or a sensor description pulled from a smell cache on the occupant's mobile communication device 110. As another example, a user may speak or interact directly with the odor mitigation system 102 or an in-dash computing system to provide a human language description and request that the odor be removed. The odor mitigation system 102 gets 904 the smell vector for the smell (e.g., see 602, 604, and 606 of FIG. 6). The odor mitigation system 102 may also request 906 information from the occupant or mobile computing device 110 about the nature and intensity of the odor (see e.g., FIGS. 6 and 7). For example, it may request a description of the odor and the occupant's perception of the odor.

The odor mitigation system 102 performs 908 mitigation to reduce or remove the odor (see e.g., FIGS. 6 and 7). The odor mitigation system 102 saves 910 the old smell vector (e.g., from 904) and get a new smell vector. The odor mitigation system 102 again requests 912 information from the mobile device about the nature and intensity of the odor. Based on the updated vector and user's response to the request 912, the odor mitigation system 102 determines 914 whether the odor is sufficiently mitigated. If the odor is not sufficiently mitigated (No at 914), the odor mitigation system 102 uses chemistry rules or other rules to change 916 the mitigation strategy based on changes in the smell vector and the resulting psychological effect on the occupant and proceeds to perform 908 mitigation. If the odor is sufficiently mitigated (Yes at 914), the odor mitigation system 102 estimates 918 the smell vector for the odor (but may omit vectors for the fragrances) and stores 920 the odor vector and the mitigation strategy in a smell cache.

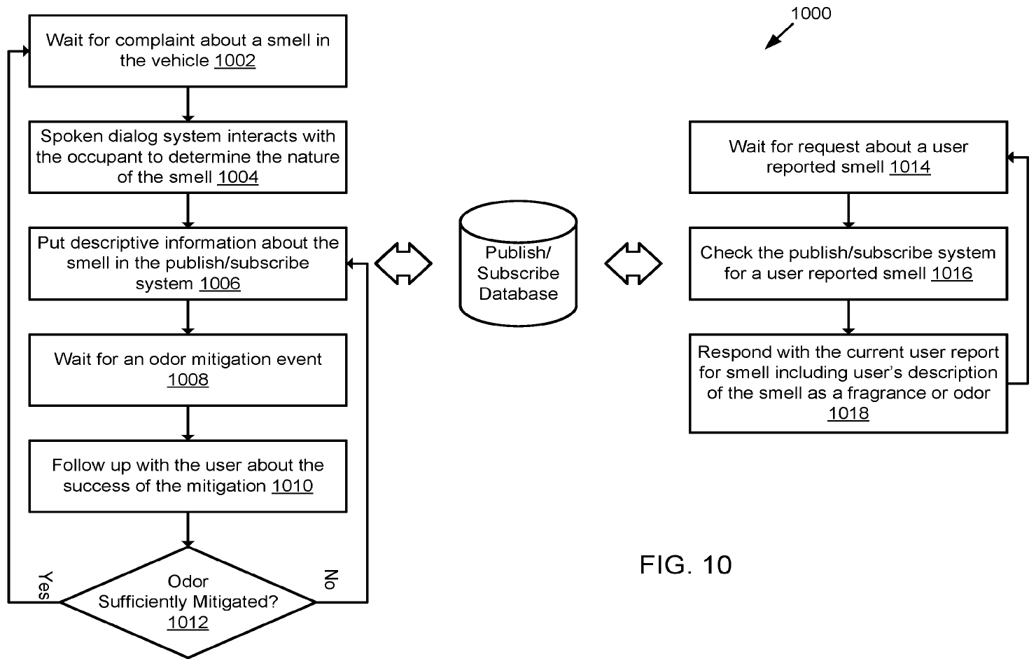

FIG. 10 is a schematic flow chart diagram illustrating a method 1000 for interaction between an odor mitigation system and a mobile communication device of an occupant, according to one embodiment. The method 100 may be performed by an odor mitigation system 102 and a mobile communication device 110, for example. For example, the mobile communication device may determine individually if the smell is an odor or fragrance to a user.

An odor mitigation system 102 may wait 1002 for a complaint about a smell in the vehicle. The complaint may come from a mobile communication device 110 or directly from a user via an audio system or speaker of the vehicle. The odor mitigation system 102 may use a spoken dialog system (of the vehicle or a mobile communication device 110) to interacts 1004 with the occupant to determine the nature of the smell. For example, the odor mitigation system 102 may get terms or a description from the user describing the smell, whether it is an odor or fragrance, and/or how strong it is. The odor mitigation system 102 places 1006 the descriptive information about the smell in the publish/subscribe system (e.g., a publish/subscribe database stored by the odor mitigation system 102). The odor mitigation system 102 waits for the occurrence of an odor mitigation event 1008, such as the performance of part or all of a mitigation procedure. The odor mitigation system 102 follows up 1010 with the user about the success of mitigation 1010. For example, the odor mitigation system 102 may receive a verbal response or response from a mobile communication device 110 indicating whether the user perceives an improvement, decay, or maintenance in the strength of the odor. If the odor is not sufficiently mitigated (No at 1012) the odor mitigation system 102 may return to placing 1006 descriptive information in the publish/subscribe system and waiting 1008 for a mitigation event. If the odor is sufficiently mitigated (Yes at 1012), the odor mitigation system 102 may wait for additional complaints about smells, if any.

An occupant's mobile communication device 110 may access information within the publish/subscribe database to respond to queries or vote on the need for mitigation. A mobile communication device 110 waits 1014 for a request about a user reported smell and checks 1016 the publish/subscribe system for a user reported smell. If there is/are user reported smells, the mobile communication device 110 responds 1018 with the current user report for smell including a user's description of the smell as a fragrance or odor. The mobile communication device 110 may wait 1014 for and respond 1018 to additional requests as needed.

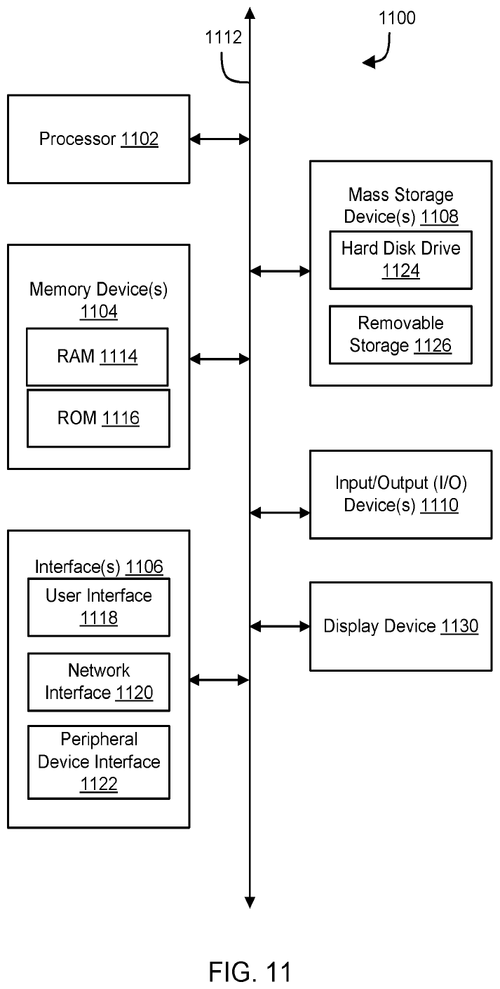

Referring now to FIG. 11, a block diagram of an example computing device 1100 is illustrated. Computing device 1100 may be used to perform various procedures, such as those discussed herein. In one embodiment, the computing device 1100 can function as an odor mitigation system 102, mobile communication device 110, or the like. Computing device 1100 can perform various monitoring functions as discussed herein, and can execute one or more application programs, such as the application programs or functionality described herein. Computing device 1100 can be any of a wide variety of computing devices, such as a desktop computer, in-dash computer, vehicle control system, a notebook computer, a server computer, a handheld computer, tablet computer and the like.

Computing device 1100 includes one or more processor(s) 1102, one or more memory device(s) 1104, one or more interface(s) 1106, one or more mass storage device(s) 1108, one or more Input/Output (I/O) device(s) 1110, and a display device 1130 all of which are coupled to a bus 1112. Processor(s) 1102 include one or more processors or controllers that execute instructions stored in memory device(s) 1104 and/or mass storage device(s) 1108. Processor(s) 1102 may also include various types of computer-readable media, such as cache memory.

Memory device(s) 1104 include various computer-readable media, such as volatile memory (e.g., random access memory (RAM) 1114) and/or nonvolatile memory (e.g., read-only memory (ROM) 1116). Memory device(s) 1104 may also include rewritable ROM, such as Flash memory.

Mass storage device(s) 1108 include various computer readable media, such as magnetic tapes, magnetic disks, optical disks, solid-state memory (e.g., Flash memory), and so forth. As shown in FIG. 11, a particular mass storage device is a hard disk drive 1124. Various drives may also be included in mass storage device(s) 1108 to enable reading from and/or writing to the various computer readable media. Mass storage device(s) 1108 include removable media 1126 and/or non-removable media.

I/O device(s) 1110 include various devices that allow data and/or other information to be input to or retrieved from computing device 1100. Example I/O device(s) 1110 include cursor control devices, keyboards, keypads, microphones, monitors or other display devices, speakers, printers, network interface cards, modems, and the like.

Display device 1130 includes any type of device capable of displaying information to one or more users of computing device 1100. Examples of display device 1130 include a monitor, display terminal, video projection device, and the like.

Interface(s) 1106 include various interfaces that allow computing device 1100 to interact with other systems, devices, or computing environments. Example interface(s) 1106 may include any number of different network interfaces 1120, such as interfaces to local area networks (LANs), wide area networks (WANs), wireless networks, and the Internet. Other interface(s) include user interface 1118 and peripheral device interface 1122. The interface(s) 1106 may also include one or more user interface elements 1118. The interface(s) 1106 may also include one or more peripheral interfaces such as interfaces for printers, pointing devices (mice, track pad, or any suitable user interface now known to those of ordinary skill in the field, or later discovered), keyboards, and the like.

Bus 1112 allows processor(s) 1102, memory device(s) 1104, interface(s) 1106, mass storage device(s) 1108, and I/O device(s) 1110 to communicate with one another, as well as other devices or components coupled to bus 1112. Bus 1112 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1100, and are executed by processor(s) 1102. Alternatively, the systems and procedures described herein can be implemented in hardware, or a combination of hardware, software, and/or firmware. For example, one or more application specific integrated circuits (ASICs) or a system on a chip (SoC) can be programmed to carry out one or more of the systems and procedures described herein.

### EXAMPLES

The following examples pertain to further embodiments.

Example 1 is a method for mitigating odors that includes detecting a known smell using on one or more odor sensors in a vehicle. The method includes determining whether the known smell is agreeable to one or more passengers of the vehicle. The method includes mitigating the known smell using one or more odor control devices if the known smell is not agreeable to the one or more passengers of the vehicle.

In Example 2, the determining whether the known smell is agreeable to the one or more passengers of Example 1 includes receiving an indication from a mobile device for each of the one or more passengers.

In Example 3, the receiving the indication of Example 2 includes one or more of a preference of a specific passenger stored on the mobile device or a response by the passenger to a query about the known smell.

In Example 4, the method of any of Examples 1-3 further includes identifying a mitigation procedure for mitigating the known smell, wherein identifying the mitigation procedure includes identifying the known smell in a mitigation database, the mitigation database indicating a mitigation procedure for the known smell.

In Example 5, the mitigation database of Example 4 includes one or more of: a database specific to the vehicle, wherein the database specific to the vehicle indicates adjustments to mitigation procedures based on specific attributes of the vehicle; and a shared database shared by a plurality of vehicles, wherein the shared database matches one or more mitigation procedures from another vehicle with the known smell.

In Example 6, the detecting the known smell in any of Examples 1-5 includes matching signals or parameters detected by the one or more odor sensors with attributes of a smell logged in a smell database.

In Example 7, the detecting the known smell in any of Examples 1-6 includes matching information from the one or more odor sensors with a description provided by a human.

In Example 8, the one or more odor sensors in any of Examples 1-7 includes at least one electronic nose and wherein the one or more odor control devices include one or more of a window controller, an HVAC circulation controller, an air filter, a fragrance source, and a chemical source including a chemical for reacting with a cause of the known smell.

Example 9 is computer readable storage media storing instructions that, when executed by one or more processors, cause the one or more processors to implement a method or realize a system or apparatus as in any of Examples 1-8.

Example 10 is a system or device that includes means for implementing a method or realizing a system or apparatus as in any of Examples 1-9.

In the above disclosure, reference has been made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium, which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, an in-dash vehicle computer, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. The terms “modules” and “components” are used in the names of certain components to reflect their implementation independence in software, hardware, circuitry, sensors, or the like. As one skilled in the art will appreciate, components may be referred to by different names. This document does not intend to distinguish between components that differ in name, but not function.

It should be noted that the sensor embodiments discussed above may comprise computer hardware, software, firmware, or any combination thereof to perform at least a portion of their functions. For example, a sensor may include computer code configured to be executed in one or more processors, and may include hardware logic/electrical circuitry controlled by the computer code. These example devices are provided herein purposes of illustration, and are not intended to be limiting. Embodiments of the present disclosure may be implemented in further types of devices, as would be known to persons skilled in the relevant art(s).

At least some embodiments of the disclosure have been directed to computer program products comprising such logic (e.g., in the form of software) stored on any computer useable medium. Such software, when executed in one or more data processing devices, causes a device to operate as described herein.

While various embodiments of the present disclosure have been described above, it should be understood that they have been presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents. The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

1. A method comprising:
detecting a known smell using on one or more odor sensors in a vehicle;
determining whether the known smell is agreeable to one or more passengers of the vehicle; and
mitigating the known smell using one or more odor control devices if the known smell is not agreeable to the one or more passengers of the vehicle.

2. The method of claim 1, wherein determining whether the known smell is agreeable to the one or more passengers comprises receiving an indication from a mobile device for each of the one or more passengers.

3. The method of claim 2, wherein receiving the indication comprises receiving one or more of: a preference of a specific passenger stored on the mobile device; or a response by the passenger to a query about the known smell.

4. The method of claim 1, further comprising identifying a mitigation procedure for mitigating the known smell, wherein identifying the mitigation procedure comprises identifying the known smell in a mitigation database, the mitigation database indicating a mitigation procedure for the known smell.

5. The method of claim 4, wherein the mitigation database comprises one or more of:
a database specific to the vehicle, wherein the database specific to the vehicle indicates adjustments to mitigation procedures based on specific attributes of the vehicle; and
a shared database shared by a plurality of vehicles, wherein the shared database matches one or more mitigation procedures from another vehicle with the known smell.

6. The method of claim 1, wherein detecting the known smell comprises matching signals or parameters detected by the one or more odor sensors with attributes of a smell logged in a smell database.

7. The method of claim 1, wherein detecting the known smell comprises matching information from the one or more odor sensors with a description provided by a human.

8. A system comprising:
one or more odor sensors in a vehicle;
one or more odor control devices for modifying smells within the vehicle;
an odor detection component configured to detect a known smell based on the one or more odor sensors;
an agreeableness component configured to determine whether the known smell is agreeable to one or more passengers of the vehicle; and
a mitigation component configured to control the one or more odor control devices to mitigate the known smell if the known smell is not agreeable to the one or more passengers of the vehicle.

9. The system of claim 8, wherein the agreeableness component determines whether the known smell is agreeable to the one or more passengers based on an indication received from a mobile device for each of the one or more passengers.

10. The system of claim 9, wherein the indication comprises one or more of: a preference of a specific passenger stored on the mobile device; or a response by the passenger to a query about the known smell.

11. The system of claim 8, wherein the mitigation component is further configured to identify a mitigation procedure for mitigating the known smell, wherein the mitigation component identifies the mitigation procedure by identifying the known smell in a mitigation database, the mitigation database indicating a mitigation procedure for the known smell.

12. The system of claim 11, wherein the mitigation database comprises one or more of: a database specific to the vehicle, wherein the database specific to the vehicle indicates adjustments to mitigation procedures based on specific attributes of the vehicle; and a shared database shared by a plurality of vehicles, wherein the shared database matches one or more mitigation procedures from another vehicle with the known smell.

13. The system of claim 8, wherein the one or more odor sensors comprise at least one electronic nose and wherein the one or more odor control devices comprise one or more of a window controller, an HVAC circulation controller, an air filter, a fragrance source, and a chemical source comprising a chemical for reacting with a cause of the known smell.

14. The system of claim 8, wherein the odor detection component detects the known smell by matching signals or parameters from the one or more odor sensors with attributes of a smell logged in a smell database.

15. The system of claim 8, wherein the odor detection component matches information from the one or more odor sensors with a description provided by a human, wherein the agreeableness component communicates the description to the one or more passengers.

16. Non-transitory computer readable storage media storing instructions that, when executed by one or more processors, cause the one or more processors to:
detect a known smell based on the one or more odor sensors;
determine whether the known smell is agreeable to one or more passengers of the vehicle; and
control the one or more odor control devices to mitigate the known smell if the known smell is not agreeable to the one or more passengers of the vehicle.

17. The computer readable storage media of claim 16, wherein the instructions cause the one or more processors to determine whether the known smell is agreeable to the one or more passengers based on an indication received from a mobile device for each of the one or more passengers.

18. The computer readable storage media of claim 17, wherein the indication comprises one or more of: a preference of a specific passenger stored on the mobile device; or a response by the passenger to a query about the known smell.

19. The computer readable storage media of claim 16, wherein the instructions further cause the one or more processors to identify a mitigation procedure for mitigating the known smell, wherein the instructions cause the one or more processors to identify the mitigation procedure by identifying the known smell in a mitigation database, the mitigation database indicating a mitigation procedure for the known smell.

20. The computer readable storage media of claim 19, wherein the mitigation database comprises one or more of:
a database specific to the vehicle, wherein the database specific to the vehicle indicates adjustments to mitigation procedures based on specific attributes of the vehicle; and
a shared database shared by a plurality of vehicles, wherein the shared database matches one or more mitigation procedures from another vehicle with the known smell.
